(19) United States
(12) Patent Application Publication
(10) Pub. No.: US 2014/0088403 A1
(43) Pub. Date: Mar. 27, 2014

(54) PATIENT COUCH FOR COMBINED MAGNETIC RESONANCE AND PET EXAMINATION

(71) Applicant: SIEMENS AKTIENGESELLSCHAFT, Munich (DE)

(72) Inventor: Patrick GROSS, Buckenhof (DE)

(73) Assignee: SIEMENS AKTIENGESELLSCHAFT, Munich (DE)

(21) Appl. No.: 14/036,206

(22) Filed: Sep. 25, 2013

(30) Foreign Application Priority Data
Sep. 26, 2012 (DE) 102012217439.7

(51) Int. Cl. A61B 5/055; A61B 5/00

(52) U.S. Cl. CPC A61B 5/0555; A61B 5/0035
USPC 600/411; 5/601

(57) ABSTRACT

A patient couch is disclosed, for a combination of positron emission tomography and magnetic resonance tomography device (PET/MRT device), for presenting images of an examination object in an examination room. A system, including the patient couch and a combination of positron emission tomography and magnetic resonance tomography device, is also disclosed. In an embodiment, the patient couch includes a reclining surface for a patient, which is designed to only insignificantly attenuate radiation intensity originating from a positron decay when it passes therethrough.

### PRIORITY STATEMENT

The present application hereby claims priority under 35 U.S.C. §119 to German patent application numbers DE 102012217439.7 filed Sep. 26, 2012, the entire contents of which are hereby incorporated herein by reference.

### FIELD

At least one embodiment of the invention generally relates to a patient couch for a combination of positron emission tomography and magnetic resonance tomography device (PET/MRT device) for presenting images of an examination object in an examination room. Furthermore, at least one embodiment of the invention generally relates to a system comprising a patient couch and a combination of positron emission tomography and magnetic resonance tomography device.

### BACKGROUND

Magnetic resonance tomography (MRT) and Positron emission tomography (PET) are currently essential methods for the precise diagnosis of many diseases and health disorders. It is herewith possible to accurately three-dimensionally image affected organs and organ parts and furthermore track the physiological and biochemical processes in the affected organs or organ parts even on a molecular level.

The strength of the MRT lies in the exact imageability of many organs with the greatest local resolution. In comparison with computed tomography (CT), this method manages without potentially harmful ionizing radiation. The power of PET in contrast lies above all in the functional imaging, i.e. in the imaging of biochemical and physiological processes. PET nevertheless has a relatively poor local resolution of for instance approximately 5 mm, which cannot be increased any further without additional radiation exposure. With a combination of both methods, the high local resolution of the MRT with the functional information from the PET can be used for an even more exact diagnosis.

The unexamined application DE 10 2005 015 070 A1 discloses a combined positron emission tomography and magnetic resonance tomography device for presenting images of an examination object in an examination room using a positron emission tomography device, which comprises a device part assigned to the examination room and a first evaluation unit for evaluating the electrical signals for a positron emission tomography image of the examination object. The device part in this case includes a gamma beam detector with an assigned electronics unit.

Furthermore, the combined device includes a magnetic resonance tomography device and a second evaluation unit for evaluating the magnetic resonance signals for a magnetic resonance image of the examination object. The magnetic resonance device in this case comprises a high frequency antenna facility and a gradient coil system, wherein the high frequency antenna facility is arranged closer to the examination room than the gradient coil system.

Furthermore, the magnetic resonance device comprises a high frequency shield arranged between the gradient coil system and the high frequency antenna facility. The positron emission tomography device part is in this case arranged between the high frequency shield and the high frequency antenna facility.

Modern magnetic resonance systems, such as are described in the unpublished application DE 10 2009 036 939 A1, the entire contents of which are hereby incorporated herein by reference, generally operate with a number of different antennas (subsequently also referred to as coils) for emitting high frequency pulses for nuclear resonance excitation and/or for receiving induced magnetic resonance signals. A magnetic resonance system frequently possesses a larger, so-called body coil which is generally permanently built into the device, also known as body coil, as well as several small surface coils, also known as local coils. Surface coils in contrast to body coils are used to record more detailed images of body parts and/or organs of a patient, which are located relatively close to the body surface. To this end, surface coils are applied to the point on the patient at which a region to be examined is located. The coil parts can also additionally possess electrical connections, realized for instance by plug-in contacts.

In the case of computed tomography systems based on x-ray radiation, and also in the case of MRT and PET, it is usual to position the patient to be examined on patient couches which are only supported at one end. In this way it is possible to completely remove the patient couch from the examination device, to position the patient and then to introduce the couch with the patient from one side into the examination device. With magnetic resonance tomography, a patient couch no longer interferes with the examination, provided it does not influence the magnetic field, comprise large-area metallic structures, which prevent high frequency propagation or comprise materials with magnetic resonances in a frequency range which is relevant to the examination object, such as for instance the resonance frequencies for hydrogen in water or fat.

For an undisturbed imaging process, positron emission tomography is dependent on the radiation emitted by the examination object reaching the detectors unattenuated as far as possible in all directions, so that an undisturbed three-dimensional imaging process can take place. With the combination of a computed tomography with a PET based on x-ray radiation, this does not represent any obstacle, since the CT is also dependent on as undisturbed an irradiation of the examination object with the x-ray radiation as possible. The local coils have nevertheless proven themselves with regard to improving the imaging during the magnetic resonance tomography. The local coils require metallic feed lines from HF transmitters and/or receivers, which significantly attenuate the radiation of the PET. With the afore-described patient couch with a support at only one end, it is necessary with the described procedure in the prior art to route feed lines to local coils at an end of the patient couch opposing the supported end along the entire patient couch so that these feed lines interfere with the imaging for the entire patient.

### SUMMARY

A patient couch is provided, which enables improved imaging in a combined positron emission and magnetic resonance tomography system.

An embodiment of the patient couch comprises a first and a second end, which are arranged at opposite ends of the couch along a longitudinal direction. A reclining surface for a patient extends between the first end and the second end. In one region of the reclining surface, the patient couch is configured to only insignificantly attenuate a radiation intensity originating from a positron decay in all directions at right angles to the longitudinal direction when it passes through the patient couch. In one embodiment, the reclining surface attenuates the radiation intensity at most by half.

An inventive system is also disclosed, comprising an inventive patient couch and a combined magnetic resonance and positron emission tomography system. In at least one embodiment, the magnetic resonance tomography system comprises a field magnet with a feedthrough, wherein the patient couch is arranged partially in the feedthrough, and wherein a distance between the first end and the second end of the patient couch is designed such that the first end and the second end are not arranged within the feedthrough during an examination.

### BRIEF DESCRIPTION OF THE DRAWINGS

The afore-described properties, features and advantages of this invention as well as the manner in which they are achieved become clearer and easier to understand in conjunction with the following description of the example embodiments, which are explained in more detail in conjunction with the drawings, in which;

FIG. 1 shows a schematic side view in the partial cross-section of a combined positron emission and magnetic resonance tomography system having a patient couch from the prior art and

FIG. 2 shows a schematic side view in the partial cross-section of a combined positron emission and magnetic resonance tomography system having a patient couch of an embodiment.

Identical elements are provided with identical reference characters in the figures respectively.

### DETAILED DESCRIPTION OF THE EXAMPLE EMBODIMENTS

The present invention will be further described in detail in conjunction with the accompanying drawings and embodiments. It should be understood that the particular embodiments described herein are only used to illustrate the present invention but not to limit the present invention.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

Specific structural and functional details disclosed herein are merely representative for purposes of describing example embodiments of the present invention. This invention may, however, be embodied in many alternate forms and should not be construed as limited to only the embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

An embodiment of the patient couch comprises a first and a second end, which are arranged at opposite ends of the couch along a longitudinal direction. A reclining surface for a patient extends between the first end and the second end. In one region of the reclining surface, the patient couch is configured to only insignificantly attenuate a radiation intensity originating from a positron decay in all directions at right angles to the longitudinal direction when it passes through the patient couch. In one embodiment, the reclining surface attenuates the radiation intensity at most by half.

An inventive system is also disclosed, comprising an inventive patient couch and a combined magnetic resonance and positron emission tomography system. In at least one embodiment, the magnetic resonance tomography system comprises a field magnet with a feedthrough, wherein the patient couch is arranged partially in the feedthrough, and wherein a distance between the first end and the second end of the patient couch is designed such that the first end and the second end are not arranged within the feedthrough during an examination.

The patient couch of at least one embodiment advantageously only marginally attenuates the radiation generated by a positron decay and in this way, with a minimal dose for the patient, enables a comparable imaging performance of the position emission tomography to be achieved or an improved imaging quality to be achieved with the same dose.

Advantageous developments of the invention are specified in the subclaims.

In one possible embodiment of the patient couch, an electrical feed line is arranged at the first end or the second end. In this way the electrical feed line does not extend between the first end and the second end along the reclining surface.

This advantageously enables local coils to be arranged on the patient in a combined magnetic resonance and positron emission tomography system, wherein the feed line to the coils come from the respective ends of the couch without the feed lines passing through the feedthrough of the field magnet and along the patient, for instance from a foot end of the couch to a local coil on the head. With a local coil on the head of the patient, the feed line on the patient couch inventively proceeds from the head end and does not lie along the body. The number and the length of feed lines in the examination region, which extend to a maximum along the body of the patient, is reduced. If the MRT and the PET take place at the same time in a combined apparatus, the interfering absorption of the gamma quanta generated by the positrons by the feed lines is advantageously reduced and the imaging of the PET is improved.

In one embodiment, the patient couch comprise a supporting device at the first end for maintaining a distance between the reclining surface and a supporting surface and comprises a connecting element for connecting the electrical feed line at the second end.

With this embodiment, it is advantageously possible to only connect a feed line to the connecting element if the patient is arranged in the feedthrough. This avoids the feed line having to pass through the feedthrough. Particularly if the patient couch has no second support, so that the reclining surface is self-supporting at the second end, it is then possible to introduce the patient couch into the feedthrough, by the second end being introduced into the feedthrough and then the patient couch being moved toward the magnet unit until the first end is positioned directly adjacent to the magnet unit. In this way the feed line can be easily plugged onto the connecting device if the second end has left the feedthrough at the opposite end.

In an example embodiment, a distance between the first end and the second end is greater than an extent of the feedthrough of the field magnet in the longitudinal direction.

In one embodiment, the distance between the first end and the second end is greater than 2 m.

A distance of this type advantageously ensures that the feed lines do not pass unnecessarily into an examination region. With a combined PET and MRT, the detectors for the PET radiation are arranged in the feedthrough of the field magnet, so that both examinations can take place at the same time. In this way the feed lines are located outside of the examination region in the feedthrough. With a distance of greater than 2 m, this also ensures that the entire body of the (majority of) patients can advantageously be examined without a feed line negatively affecting the PET imaging in the examination region.

In one embodiment, the patient couch comprises at least one movement device along the longitudinal direction, wherein movement devices are arranged, for example, at the first end and the second end. The movement devices enable the patient on the patient couch to be brought into the examination region in the feedthrough and also to be moved there in order to examine the entire body or larger areas.

It is advantageous here if the movement devices, for instance drives and linear traction, are arranged at the ends. As a result, these solid parts are not located in the examination region and do not contribute to the absorption of gamma quanta generated by the positrons, as a result of which the imaging quality of the PET is improved.

In one embodiment, the patient couch comprises a plurality of supporting devices for maintaining a distance from a supporting surface, wherein the supporting devices are arranged at the first end and the second end.

Patient couches from the prior art usually comprise supports at one end of the patient couch, which allows the couch with the patient to be removed from the tomography device, the patient to be positioned thereupon and the couch with the patient to be introduced from one end into the tomography device. The self-supporting couch on one side nevertheless requires a rigid and correspondingly solid construction. In one embodiment of the inventive patient couch, supports are located at the first and the second end of the patient couch, which arranges the patient couch at the height of the feedthrough of the field magnet. The two-sided support advantageously enables the construction of the patient couch to be embodied to be weaker and thus less solid, thereby resulting in a lower absorption of gamma quanta and thus also in an improved PET imaging process.

In one embodiment of the inventive system, the positron emission tomography device comprises radiation detectors, wherein the radiation detectors are arranged within the feedthrough. In one embodiment of the system, the feed lines are arranged outside of the feedthrough.

By the radiation detectors being arranged in the feedthrough of the field magnet and the feed lines being arranged outside thereof, the feed lines do not lie between the patient and/or examination object and the radiation detectors. The gamma radiation of the PET is not attenuated by the feed line, thereby advantageously improving the quality of the imaging of the PET. Furthermore, the examination region is excited in the feedthrough by the local coils and/or a body coil with electromagnetic waves in the radio frequency range. A metallic feed line in the feedthrough simultaneously acts as an antenna and discharges to its outer conductor, if this is a coaxial line, high frequency energy from the examination region. To prevent this, in the prior art sheath wave filters are to be provided on the feed lines. By the feed lines not being arranged in the feedthrough and thus not receiving any HF energy, it is advantageously possible to dispense with the sheath wave filter.

In one embodiment of the system, the movement device are designed to move the reclining surface out of the feedthrough.

Particularly with the embodiment with supporting devices at the first and second end, it is advantageously possible in such cases to prepare the patient outside of the feedthrough on the reclining surface and then to move the same into the feedthrough, where they are still difficult for operating personnel to access.

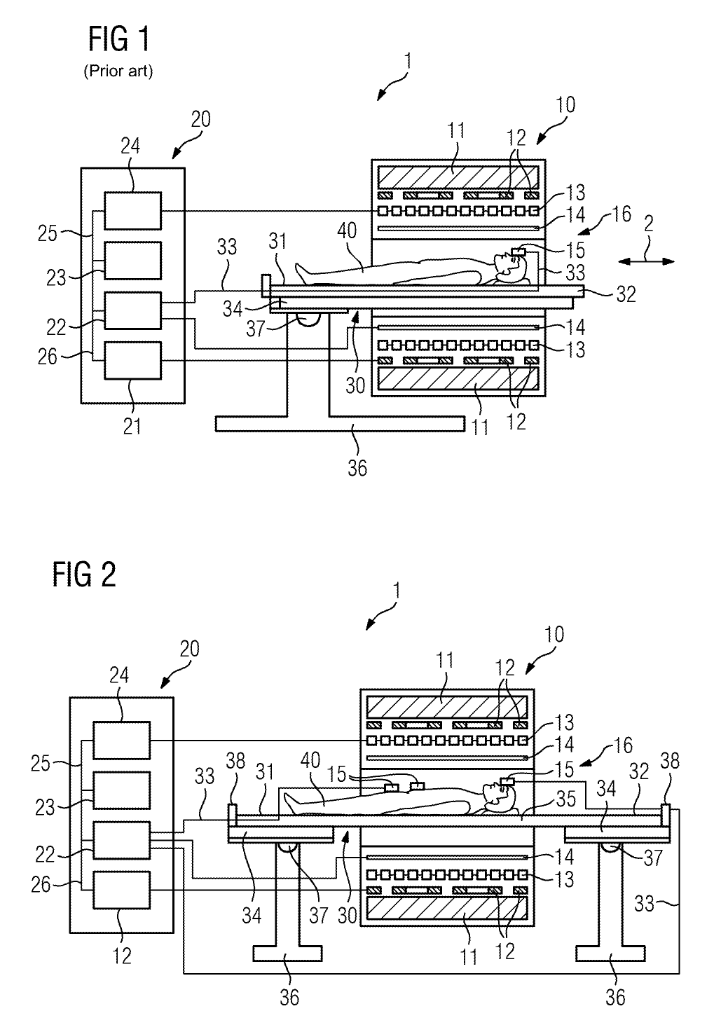

FIG. 1 shows a combined positron emission and magnetic resonance tomography system 1 with a patient couch 30 from the prior art.

The magnet unit 10 comprises a field magnetic 11 which generates a static magnetic field BO for aligning the nuclear spin of test samples and/or patients 40 in an examination region. The examination region is arranged in a feedthrough 16, which extends in a longitudinal direction 2 through the magnet unit 10. The field magnet 11 is usually a superconductive magnet, which can provide magnetic fields with a magnetic flux density of up to 3 T, or even above in the case of the most modern devices. For lower field strengths, permanent magnets or electromagnets with normal conductive coils can however also be used.

Furthermore, the magnet unit 10 comprises gradient coils 12, which are designed, for spatial differentiation of the acquired imaging regions in the examination region, to superimpose variable magnetic fields on the magnetic field BO in three spatial directions. The gradient coils 12 are usually coils comprising normally conducting wires, which can generate fields in the examination region which are orthogonal to one another.

The magnet unit 10 likewise comprises a body coil 14, which is designed to irradiate a high frequency signal supplied by way of a signal line into the examination region and to receive resonance signals emitted by the patient 40 and to output the same by way of the signal line. The body coil 14 for the emission of the high frequency signal and/or the reception is preferably replaced by local coils 15, which are arranged in the feedthrough 16 close to the patient 40.

Radiation detectors 13 are also arranged in the magnet unit 10. The radiation detectors 13 preferably surround the examination region in all spatial directions and as extensively as possible in order to detect the highest possible number of gamma quanta, which is emitted during positron emission tomography by the patient 40.

It is advantageous here if as little material as possible is located between the patient 40 and the radiation detector 13, which can absorb gamma quanta and thus extends the measurement time and/or requires a higher radiation intensity and thus also a larger number of positron emitters.

The radiation detectors 13 are therefore usually arranged on a side of the field coil 11 and the gradient coils 12 facing the feedthrough 16. The body coil 14 by contrast is arranged further from the feedthrough 16 so that a shield for high frequency radiation in the radio frequency range (not shown) can be arranged between the body coil 14 and/or feedthrough and radiation detector 13. This shield prevents the radiation detectors 13 being disturbed or even destroyed by induced voltages, caused by the high frequency radiation. The shield is designed to be thin here such that it only marginally attenuates the gamma radiation.

A control unit 20 supplies the magnet unit 10 with the various signals for the gradient coils 12 and the body coil 14 and/or the local coils 15 and evaluates the received signals.

The control unit 20 thus comprises a gradient controller 21, which is designed to supply the gradient coils 12 by way of feed lines with variable currents, which provide the desired gradient fields in the examination region in a time-coordinated fashion.

Furthermore, the control unit 20 comprises a high frequency unit 22, which is designed to generate a high frequency pulse with a predetermined temporal course, amplitude and spectral power distribution in order to excite a magnetic resonance of the nuclear spin in the patient 40. Pulse powers in the range of kilowatts can be achieved in this way.

The high frequency unit 22 is also designed to evaluate high frequency signals in respect of amplitude and phase received from the body coil 14 or a local coil 15 and fed via a signal line 33 to the high frequency unit 22. This involves in particular high frequency signals, which nuclear spin in the patient 40 emits as a response to the excitation by a high frequency pulse in the magnetic field BO and/or in a resulting magnetic field from a superimposition of BO and gradient fields.

The control unit 20 furthermore comprises a PET unit 24, which is designed to receive the signals of the radiation detectors 13 by way of one or more signal lines and to evaluate the same in respect of their temporal and spatial correlation in order to determine the place of origin of a positron decay event in the patient 40.

Furthermore, the control unit 20 comprises a controller 23, which is designed to perform the temporal coordination of the activities of the gradient activation 21 and the high frequency unit 22. To this end, the controller 23 is connected to the other units 21, 22, 24 by way of a signal bus 25 and exchanges signals with them. The controller 23 is designed to receive and process signals evaluated by the high frequency unit 22 from the patient 40 or to predetermine and temporally coordinate pulse and signal forms to the gradient activation 22 and the HF pulse generation unit 23. The controller 23 is also able to correlate the data received by the PET unit 24 and the high frequency unit 22 so that a magnetic resonance measurement is present during a decay event of a positron in the body of the patient 40 and the results of both examination methods are reproduced in a mutual spatial representation. The creation of such images can either take place by the controller 23 itself or by processing the acquired data in a further image processing unit (not shown).

With a combined positron emission and magnetic resonance tomography system, the patient 40 is arranged on a patient couch 30. These patient couches 30 are already known from magnetic resonance tomography. On account of the restricted space in the feedthrough 16, the proven approach in magnetic resonance tomography has been to position and prepare the patient 40 outside of the magnet unit 10 on the patient couch 30. Subsequently, the patient 40 is moved for examination purposes toward the magnet unit 10 and is introduced into the feedthrough 16. For this purpose, the patient couch 30 comprises a first support 36, which is arranged below a first end 31 of the patient couch 30. To ensure that the support 36 is able to hold the patient couch 30 in a horizontal position, it usually comprises a foot, which extends along the patient couch 30. In order to move the patient couch 30, the foot can also comprise movement devices, such as rollers. No constructive element is arranged between the base and the patient couch in addition to the support 36 so that the patient couch can be introduced into the feedthrough 16 of the field magnet 11 up to the first end 31.

It is however also conceivable that the patient couch 30 is held in a horizontal position by fastening the supports 36 to the floor or in a guide on the floor. Furthermore, the patient couch 30 can comprise devices 34 for moving it in the longitudinal direction 2. Linear rail systems 34 are shown in FIG. 1, which connect the supports 36 to the patient couch 30 in a moveable manner so that the patient couch can be moved along the longitudinal direction 2. To this end, the linear rail system comprises a drive 37, which allows the patient couch 30 to be moved in the longitudinal direction 2, controlled by an operating person or also by the controller 23, so that it is also possible to examine regions of the patient which have a greater extent than the examination region in the feedthrough 16.

Since the patient 40 on the patient couch 30 is prepared for the examination outside of the magnet unit 10 and the patient couch 30 with the patient 40 is then introduced into the feedthrough 16, electrical apparatuses such as the local coils 15, which are arranged at the second end 32 of the patient couch 40 at a distance from the support 36, are connected to the control unit 20 and/or the high frequency unit 22 by way of signal lines 33. The signal line 33 extends here along the patient couch 30 to the first end 31 and during the examination is located in the feedthrough 16. In such cases the signal line 33 shades out radiation detectors 13 for instance, which are located in a line with the signal line 33 and a radiation source in the patient 40 and reduces the number of gamma quanta which reach the radiation detector 13. Since upon excitation of the nuclear spin by way of the body coil 14 the signal line 33 is also located in the electromagnetic high frequency alternating field, the signal line 33 also acts as an antenna. It is therefore also necessary to prevent high frequency energy from dissipating from the examination region and causing interferences using suitable measures, such as for instance a cable sheath filter.

FIG. 2 shows a combined positron emission and magnetic resonance tomography system 1 with a possible embodiment of the inventive patient couch 30. Here the magnet unit 10 and the control unit 20 are unchanged by comparison with FIG. 1.

The patient couch 30 shown in FIG. 2 comprises two supports 36, which are arranged at a first end 31 and a second end 32 of the patient couch 30 opposite one another in the longitudinal direction 2. The supports are designed to hold the patient couch 30 in a horizontal position such that a reclining surface 35 of the patient couch 30, which extends between the first end 31 and the second end 32 is provided to position the patient 40 thereupon, extends horizontally through the feedthrough 16. Positions of the patient couch 30 which are inclined in accordance with the alignment of the feedthrough 16 are however also conceivable.

In one embodiment, movement devices 34 are arranged between the supports 36 and the first end 31 and the second end 32 of the patient couch 30. Linear rail systems 34 are shown for instance in FIG. 2, which allow, with the aid of drives 37, the reclining surface 35 of the patient couch 30 to be moved relative to the feedthrough 16 and the magnet unit 10. In this way the patient 40 can be moved relative to the examination region in the feedthrough 16 so that examinations of the entire body are also possible. In one embodiment, the length of the reclining surface 35 of the patient couch 30 and the length of the movement devices 34 in the longitudinal direction 2 are designed such that the patient 30 can be moved entirely out of the feedthrough 16 in order to leave the patient couch 30 or vice versa the patient couch 30 can be moved far enough therefrom for the patient 40 on the reclining surface 30 of the patient couch 30 can be prepared for the examination.

In the preferred embodiment, an electrical feed line 33 is arranged at the second end 32 of the patient couch 30. This feed line can be provided for instance for the connection of a local coil 15. In one embodiment, a connecting element 38, for instance a plug-in connection 38, can also be provided so that an electrical connection can also be established between the local coil 15 and the feed line 33.

Provision is likewise made in one embodiment for an electrical feed line 33 to be arranged at the first end 31, which is provided for a local coil 15 for instance, which is arranged on another body part. A first local coil 15 can be arranged on the head of the patient 40 for instance and can be connected to a feed line 33 at the second end 32 of the patient couch, whereas a second local coil 15 can be arranged on the abdomen of the patient 40 and connected to a feed line 33 at the first end of the patient couch. The feed lines 33 are not arranged here in the feedthrough 17, but are instead routed outside of the magnet unit 10 to the control unit 20.

In one embodiment, the reclining surface 35 of the patient couch 30 is configured such that it only insignificantly attenuates the gamma radiation generated by the positron decay. In particular, the reclining surface 35 is designed to at most halve the intensity of the gamma radiation. To this end, this reclining surface 35 is only made of materials which have a low absorption coefficient in the energy range of the gamma radiation. The reclining surface 35 is preferably made from plastics or carbon fiber composite materials. Other materials, the atoms of which have a low atomic number, are suited hereto. In a preferred embodiment, provision is made here for the movement devices 34 not to extend into the region of the reclining surface 35.

In the embodiment with two supports 36 at the first end 31 and the second end 32, it is advantageous here if the statics render possible a distribution of the load by the weight of the patient 40 on two spaced supports 36, so as to embody the reclining surface 35 between the supports to be particularly thin and thus to absorb little radiation.

Provision is also made in one embodiment for the patient couch 30 only to be supported by a support 36 at a first end. In this embodiment, the reclining surface 35 of the patient couch 30 exhibits an adequate support strength for the weight of the patient 40 and at the same time a low absorption for gamma radiation due to a suitable design of the construction and selection of the materials, for instance carbon fiber composite materials of other, electrically non-conductive carbon fiber composite materials. The patient couch 30 can be easily removed from the feedthrough 16. In this embodiment, it is particularly advantageous if the connecting elements 38 are provided at the second end 32, so that a connection for instance of the local coil 15 and the feed line 33 can be quickly established.

Although the invention was illustrated and described in detail by the preferred exemplary embodiment, the invention is not restricted by the disclosed examples and other variations can be derived herefrom by the person skilled in the art without departing from the scope of protection of the invention.

What is claimed is:

1. A patient couch for combined positron emission and magnetic resonance tomography, the patient couch comprising:
a first end and a second end, arranged opposite one another on the patient couch along a longitudinal direction; and
a reclining surface for the patient, extending between the first end and the second end, the patient couch, in a region of the reclining surface, being designed to only insignificantly attenuate a radiation intensity, originating from a positron decay in all directions, at right angles to the longitudinal direction when radiation for the combined positron emission and magnetic resonance tomography passes through the patient couch.

2. The patient couch of claim 1, wherein the patient couch is designed to at most halve the radiation intensity.

3. The patient couch of claim 1, wherein an electrical feed line is arranged at the first end or the second end, and wherein the electrical feed line does not extend between the first end and the second end along the reclining surface.

4. The patient couch of claim 3, wherein the patient couch further comprises:
a supporting device at one end for maintaining a distance between the reclining surface and a supporting surface; and
a connecting element at the second end, for connecting the electrical feed line.

5. The patient couch of claim 1, wherein a distance between the first end and the second end is relatively greater than an extent of the feedthrough of a field magnet of a magnetic resonance tomography system in the longitudinal direction.

6. The patient couch of claim 1, wherein a distance between the first end and the second end is greater than 2 m.

7. The patient couch of claim 1, further comprising:
movement devices configured for movement in the longitudinal direction, wherein the movement devices are arranged at the first end and the second end.

8. The patient couch of claim 1, further comprising:
a plurality of supporting devices, arranged at the first end and the second end.

9. A system, comprising:
the patient couch of claim 1; and
a combined magnetic resonance and positron emission tomography system, a magnetic resonance tomography portion of the combined system including a field magnet with a feedthrough, wherein the patient couch is arranged partially in the feedthrough and wherein a distance between the first end and the second end of the patient couch is designed such that the first end and the second end are not arranged within the feedthrough during an examination of a patient.

10. The system of claim 9, wherein an electrical feed line is arranged at the first end or the second end, and wherein the electrical feed line does not extend between the first end and the second end along the reclining surface and wherein the feed line is arranged outside of the feedthrough.

11. The system of claim 10, wherein a positron emission tomography system portion of the combined system comprises radiation detectors and wherein the radiation detectors are arranged within the feedthrough.

12. The system of claim 9, wherein the patient couch further includes movement devices configured for movement in the longitudinal direction, wherein the movement devices are arranged at the first end and the second end and wherein the movement devices are designed to move the reclining surface out of the feedthrough.

13. The patient couch of claim 2, wherein an electrical feed line is arranged at the first end or the second end, and wherein the electrical feed line does not extend between the first end and the second end along the reclining surface.

14. The patient couch of claim 4, wherein a distance between the first end and the second end is relatively greater than an extent of the feedthrough of a field magnet of a magnetic resonance tomography system in the longitudinal direction.

15. The patient couch of claim 5, wherein a distance between the first end and the second end is greater than 2 m.

16. The patient couch of claim 14, wherein a distance between the first end and the second end is greater than 2 m.

17. The patient couch of claim 2, wherein a distance between the first end and the second end is greater than 2 m.

18. The patient couch of claim 2, further comprising:
movement devices configured for movement in the longitudinal direction, wherein the movement devices are arranged at the first end and the second end.

19. The patient couch of claim 2, further comprising:
a plurality of supporting devices, arranged at the first end and the second end.

20. The patient couch of claim 7, further comprising:
a plurality of supporting devices, arranged at the first end and the second end.

21. A system, comprising:
the patient couch of claim 2; and
a combined magnetic resonance and positron emission tomography system, the magnetic resonance tomography system including a field magnet with a feedthrough, wherein the patient couch is arranged partially in the feedthrough and wherein a distance between the first end and the second end of the patient couch is designed such that the first end and the second end are not arranged within the feedthrough during an examination of a patient.

22. The system of claim 9, wherein a positron emission tomography system portion of the combined system comprises radiation detectors and wherein the radiation detectors are arranged within the feedthrough.

23. The system of claim 10, wherein the patient couch further includes movement devices configured for movement in the longitudinal direction, wherein the movement devices are arranged at the first end and the second end and wherein the movement devices are designed to move the reclining surface out of the feedthrough.

24. The system of claim 11, wherein the patient couch further includes movement devices configured for movement in the longitudinal direction, wherein the movement devices are arranged at the first end and the second end and wherein the movement devices are designed to move the reclining surface out of the feedthrough.
